(19) United States
(12) Patent Application Publication
(10) Pub. No.: US 2015/0328047 A1
(43) Pub. Date: Nov. 19, 2015

(54) Cataract Removal Tool

(71) Applicant: Francis Y. Falck, JR., Stonington, CT (US)

(72) Inventor: Francis Y. Falck, JR., Stonington, CT (US)

(21) Appl. No.: 14/714,399

(22) Filed: May 18, 2015

Related U.S. Application Data
(60) Provisional application No. 62/000,056, filed on May 19, 2014.

(51) Int. Cl. A61F 9/007

(52) U.S. Cl. CPC A61F 9/00745

(57) ABSTRACT

A cataract emulsifying tool having a hollow shaft delivering pulsed fluid is improved by having a bend in the hollow tube or an angularly adjustable socket receiving the hollow tube. This disposes the axes of the handle and the hollow shaft at angles to each other to facilitate working over a prominent superior orbital rim or right and left angles ergonomically benefiting a surgeon.

### RELATED APPLICATIONS

This application is a non-provisional application related to provisional application No. 62/000,056 filed on 19 May 2014.

### TECHNICAL FIELD

Ophthalmological surgical instruments.

### BACKGROUND

Cataract surgery is performed using an emulsification hand piece with a disposable tip. The tip directs ultrasonically pulsed fluid against the cataract material, breaking it into small particles that are then aspirated from the eye. The current tips 10 that are commonly used are illustrated schematically in prior art FIGS. 1a, 1b and 2. These prior art tips 10 have a straight shaft extending from the handle to a straight flat opening or to an angled end 13 with a beveled opening 12. The purpose of the tip and the opening at the end is to maximize contact of the ultrasonic pulsation with the cataract material. The angle of contact or angle of incidence of the tip opening with the cataract material is critical in order to safely and efficiently emulsify the cataract material.

### PROBLEM ADDRESSED

A problem arises with eyes that are recessed relative to the bony superior orbital rim 16, which cannot maintain a proper efficient and safe angle of incidence of the hand piece relative to the cataract material. To access the cataract material, using a prior art needle, the hand piece 20 must be elevated above the superior orbital rim 16, which then places the tip opening 12 down at an inefficient unsafe angle, FIG. 2. This increases both operating time and the probability of damaging the lens capsule support structure.

Various solutions to this problem are less than ideal. To avoid the superior orbital rim 16 a surgeon may attempt a lateral approach, opposite the nose, into an eye. This requires the surgeon to use either a right or left non dominant hand maneuver, depending on whether the surgical eye is the right or left. Ergonomically this presents challenges in both handedness and equipment location. The most efficient and safest position for a surgeon to work is from the top of the patient's head directly above the eye, unless this position is blocked by the superior orbital rim. Minimizing interference of the superior orbital rim and maximizing the contact angle of the tip opening with the cataract material, especially in deep set eyes, enhances the safety and efficiency of cataract surgery.

Additionally, having the ability to use a tip that angles to the right or left, depending on whether the surgeon is right or left handed and whether it is a right or left surgical eye maximizes ergonomic control and safety of the cataract procedure.

### SUMMARY

Prior art solutions have missed the subject matter claimed because of improper analysis of the problem and failure to develop a sufficiently broad choice of remedies. The solution for cataract surgery on eyes that are relatively deeply recessed below the superior orbital rim 16 is to form an additional bend 25 in the shaft of tip 11 preferably near handle 20. This angles shaft 11 over the superior orbital rim 16, so that the open end 12 of the tip will be positioned at the appropriate angle of incidence with the cataract material. The bend 25 in the shaft tip 11 eliminates the need to place hand piece 20 in an unsafe unacceptable ergonomic position. Bend 25 optimizes cataract material removal, and creates a comfortable, safe and ergonomically effective operating position for the surgeon. Bend 25 can be used in emulsification tip designs that have a straight shaft with angle 13 or without angle 13, optimizing angle of incidence for cataract removal.

An alternative to bend 25 is an angularly adjustable socket 40 preferably arranged within handle 20. This can have the advantage of adjusting the needle up or down for surgery proceeding over the superior orbital rim and also allow lateral adjustments for ergonomic comfort of a surgeon going laterally into an eye. Either way, such a socket can dispose a needle at an adjustable angle from handle 20.

Bend 25, or a socket adjustment 40, can range from 2-45°. I have also found that a slight bend 25 at 2-5° improves the contact angle for eyes that are not deeply recessed relative to the superior orbital rim 16. Bend 25 can also be located in a mid-region of shaft of tip 11. The drawings illustrate the problem and bend angle solution. Otherwise, bends of 2-5° are small enough to become unnoticeable.

An additional bend to the right 26 for a left handed surgeon operating on a right eye and to the left 27 for a right handed surgeon operating on a left eye in the shaft of tip 11 optimizes ergonomics when operating from above the eye. Bend 26 and 27 can be from 2° to 45° . For a right handed surgeon, left bend 27 is optimum. For a left handed surgeon, right bend 26 is optimum. Bend 25 can be combined with bend 26 or bend 27.

### DRAWINGS

FIGS. 1a and 1b and are side elevational views of a prior art emulsification tip 10 with angle 13 and without angle 13.

FIG. 2 is a prior art the tip of FIG. 1 angled over the orbital rim resulting in an inefficient and unsafe contact angle.

FIG. 3 is a side elevational view of the improved tip having an additional bend 25 positioned over the orbital rim in proper contact with cataract material.

FIG. 4 is the improved tip 10 with bend 25 in side elevational view.

FIGS. 5a and 5b are top elevational views showing tip 11 with a bend to the right 26 and a bend to the left 27.

FIG. 6 is a schematic side view of a standard straight shaft or needle having an angled tip at the discharge end of a straight shaft.

FIG. 7 shows the ergonomically improved instrument with a bend 25 near handle 20.

FIG. 8 schematically shows that the instrument of FIG. 7 can have a handle angled more steeply from the shaft than is possible with a straight shaft.

FIG. 9 is a schematic prior art view of an eye showing an incision region.

FIG. 10 schematically shows a prior art straight shaft 10 approaching cataract material in the eye, without interference from a superior orbital rim.

FIG. 11 schematically shows the prior art instrument of FIG. 10 angled upward at a steeper than desirable angle because of the prominent superior orbital rim.

FIG. 12 shows the effect of an extra bend maneuvering over the prominent superior orbital rim to access the cataract material at a proper angle of attack.

FIGS. 13 and 14 schematically show an angular adjustable socket 40 in a handle 20 to achieve angular adjustment between the axes of handle 20 and shaft 10.

### DETAILED DESCRIPTION

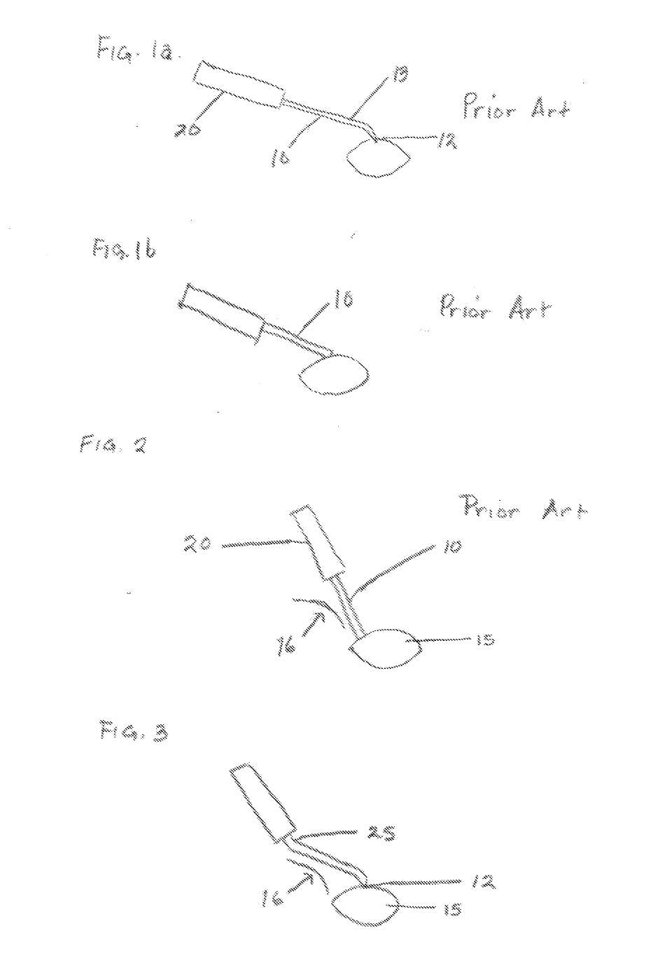

Prior art FIGS. 1a and b, and 2 illustrate the problem of a high orbital rim plane 16 of a deeply recessed eye and the difficulty of operating needle 10 at an overly steep angle to avoid the interference from the high orbital rim plane 16.

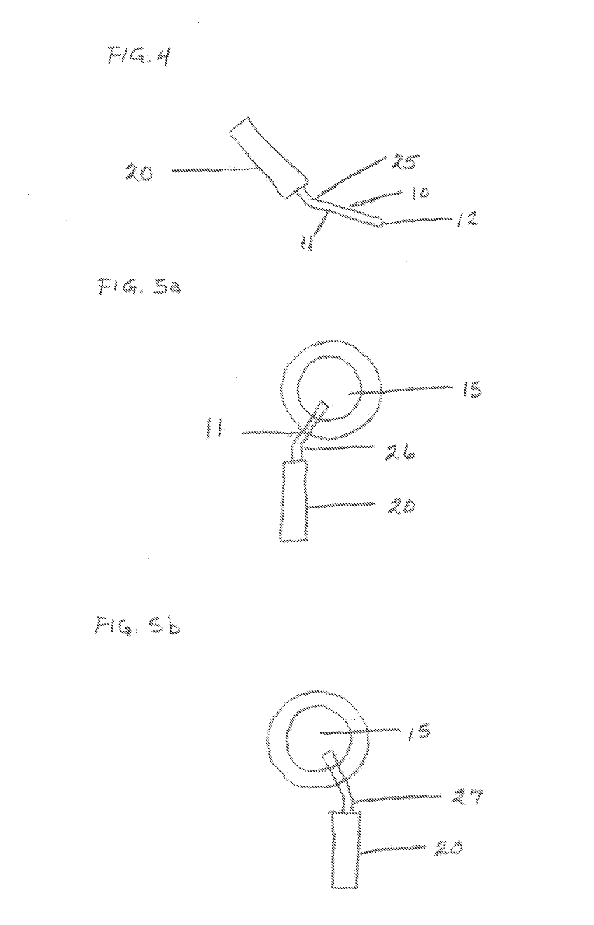

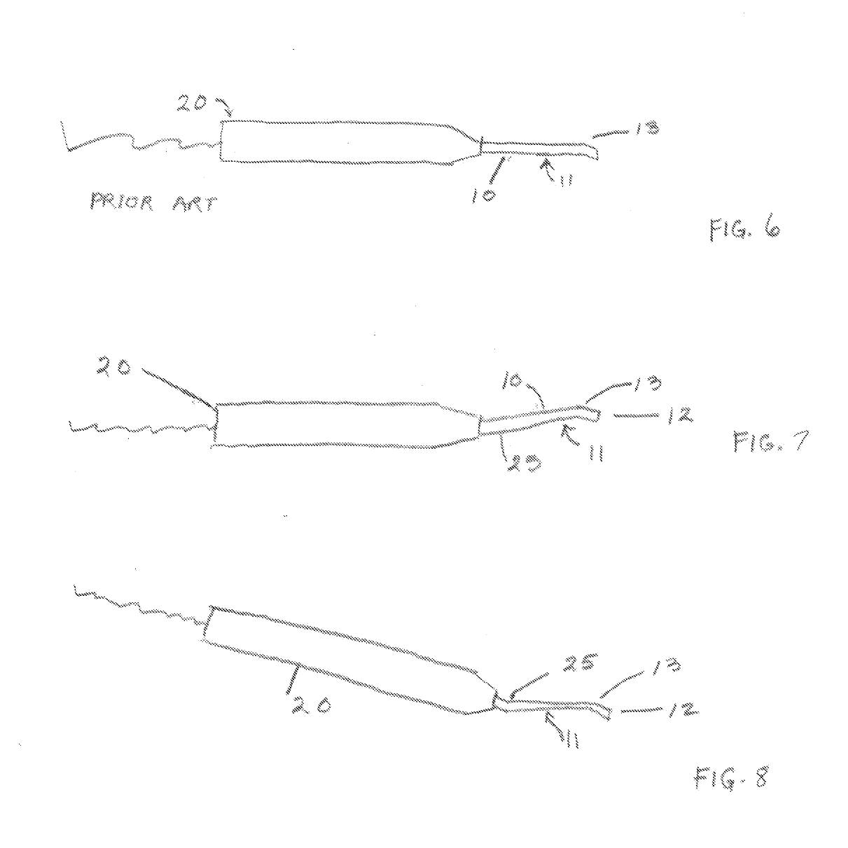

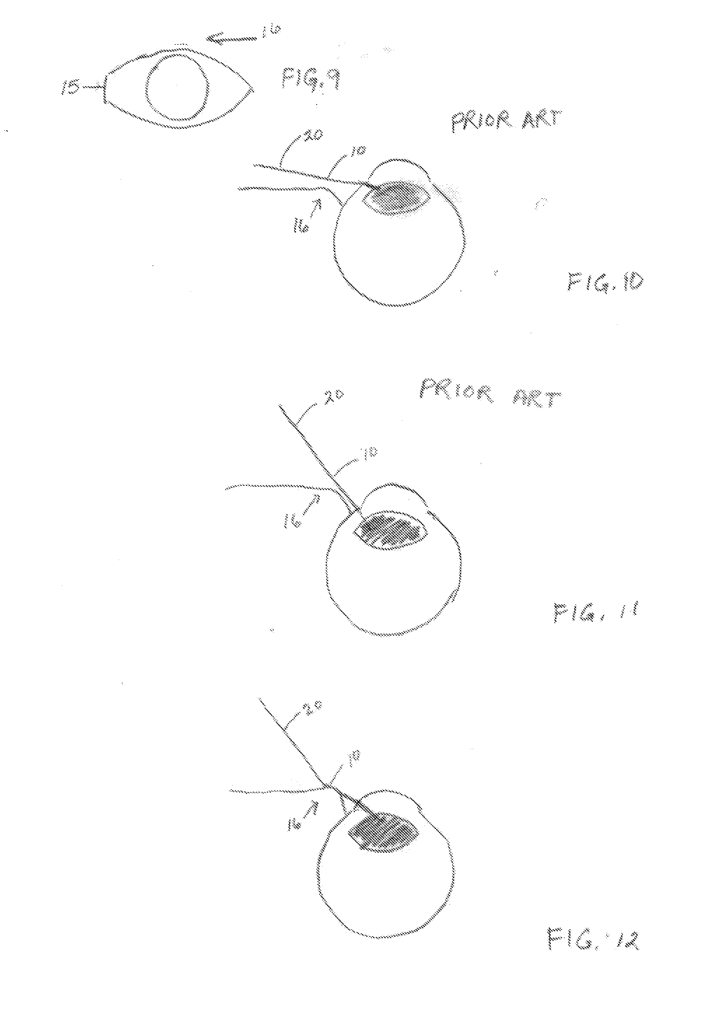

The solution making cataract removal a safer and more effective surgical procedure is illustrated in FIGS. 3-5. If the eye is not deeply recessed, as appears in an upper region of FIG. 2, the prior art needle 10 can operate effectively. A high orbital rim 16 guarding a deeply set eye requires another solution that is illustrated in FIGS. 3 and 4 where the blocking effect of the high orbital rim 16 is avoided by a bend 25 in the tip shaft 11. Bend 25 in tip shaft 11 aims needle 10 downward at a shallower angle as illustrated in FIG. 3, relative to a shaft 11 without bend 25, as illustrated in FIG. 2. With bend 25 tip opening 12 benefits by maintaining the proper contact angle with the cataract material. By maintaining the proper contact angle between tip opening 12 and the cataract material without having to place hand piece 20 in an unsafe or unacceptable position, surgery is made more efficient and safer.

Placing bend 26 or bend 27 in shaft 10 of tip 11, as illustrated in FIGS. 5a and 5b, can optimize hand ergonomics by compensating for right or left handedness. Bend 25 can be combined with bend 26 or bend 27 to compensate for a recessed eye, and right or left handedness. FIG. 6 schematically illustrates handle 20 and straight shaft 10 leading to angled tip 11. Needle 10 has a generally known bend 13 near needle tip 11.

When the embodiment of FIG. 6 encounters a prominent superior orbital rim, the straight shaft becomes difficult to work with. Handle 20 must stay clear of the superior orbital rim, and this changes the angle of attack of the pulsed fluid through tip 11. The bend 25 in shaft 10 solves this problem as shown in FIGS. 7 and 8. Bend 25 allows handle 20 to be angled upward to avoid the superior orbital rim, while maintaining an efficient angle of attack of the pulsed jet fluid passing through tip 11. Bend 25 also can angle shaft 10 downward for a similar effect.

The degree of shaft angulation to avoid a problem with the superior orbital rim is proportional to the depth of recess of the eye relative to the superior orbital rim. A practical working range for this angulation is 2° to 45°. The bend 25 can thus be made at varying angles to allow the tool to clear the superior orbital rim while delivering pulsed fluid at an efficient angle of attack. This makes the operation to remove the cataract material quicker and less dangerous than if the operation were done with a prior art device.

FIG. 9 schematically shows an eye with a preferred incision site 15. The handle 20 and shaft 10 of the prior art instrument shown in FIG. 10 is indicated by a straight line leading to the darkened cataract material. In FIG. 10, the superior orbital rim is not uncomfortably high. Conversely, the eye in FIG. 10 is not recessed relatively far below the superior orbital rim of FIG. 10. This situation calls for little or no bending of shaft 10.

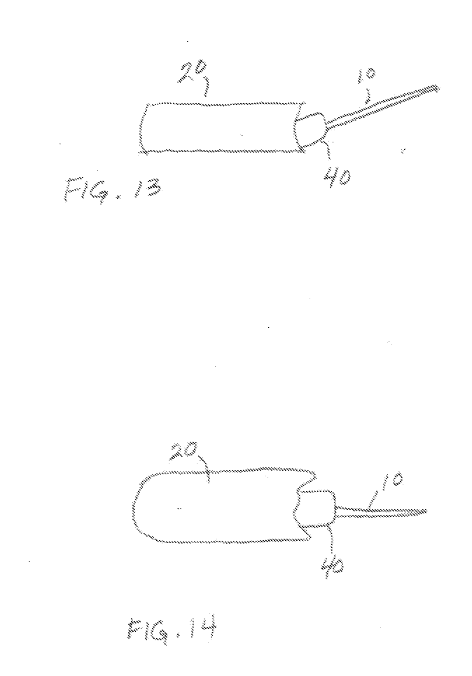

When this condition changes, as shown in FIG. 11, then the angle of attack shown by the axis line 19 of the cataract material removing tool, the steepness of the angle of attack makes the procedure difficult. The bend establishing an angle between the axes of the handle 20 and the shaft 10, as shown in FIG. 12 allows the instrument to avoid interference with the relatively high superior orbital rim so that the tip of the instrument can deliver pulsed fluid at an efficient angle of attack.

An alternative to bend 25 in shaft 10 of an emulsifying tool is shown in FIGS. 13 and 14. The bend 25 is replaced by an angularly adjustable socket 40 into which shaft 10 is threaded or otherwise attached. The angular adjustment of socket 40 can occur up or down or side-to-side in a respectively vertical or horizontal plane, so that a single embodiment of an angularly adjustable socket 40 can accommodate working down over a prominent superior orbital rim or working ergonomically for right- or left-handedness of a surgeon. The adjustability of socket 40 can be accompanied by an indicator showing the surgeon what angle is being indicated.

The adjustable socket alternative of FIGS. 13 and 14 shows that the axes of needle shaft 10 and handle 20 can be angled relative to each other throughout a range of movement. Also, the distance between the axes of handle 20 and shaft 10 puts these elements farther apart with distance from socket 40. The differences in axial angulation can adapt a single tool to accommodate adjustments for maneuvering over the superior orbital rim or adapting ergonomic solutions suitable to a surgeon. This can avoid having an inventory of needles with different bend angles. It also places control over axial angularity in the adjustment of socket 40, rather than in the disposable needle shaft 10.

The difficulty of working with the high superior orbital rim as shown in FIG. 11 is not merely an ergonomic issue for the surgeon, but it makes the whole procedure less efficient. This problem not only reduces efficiency, but can make the procedure require more time which increases the risk of lens bag rupture or some other unsafe development. The angled shaft embodiment shown in FIG. 12 keeps the discharge tip of the tool in a proper plane for making cataract surgery efficient and safe in high orbital rim and a deep set eye. The bend 25, which makes cataract removal faster, safer and more ergonomically satisfying to the surgeon is a simple matter, but countless cataract operations have occurred before applicant recognized and proposed a solution.

I claim:

1. An improvement in an emulsification tool used to direct pulsed fluid against cataract material in an eye to surgically remove cataract material from the eye, the tool having a handle, a hollow shaft, and a tip that delivers the pulsed fluid, the improvement comprising:
an axis of the shaft is disposed at an angle away from an axis of the handle so that a separation of the axes increases with distance from the handle;
the separation of the axes is oriented to the shaft to clear a superior orbital rim of an eye while allowing the tip to aim at the cataract material of the eye;
the separation of the axes also being arranged to dispose the tip at an effective angle relative to the cataract material while holding the shaft clear of the superior orbital rim; and
the angle between the axes being in a range of 2° to 45°, depending on the contour of the superior orbital rim.

2. The improvement of claim 1 wherein the angle between the axes is established by a bend in the shaft.

3. The improvement of claim 1 wherein the angle between the axes is established by a socket in the handle.

4. The improvement of claim 3 wherein the socket is angularly adjustable relative to the handle axis.

5. A tool having a hollow needle shaft extending from a handle to receive and deliver pulsed fluid to break up cataract material in an eye, the tool comprising:
the shaft being disposed at an angle to the handle;
the angle being sufficient to allow the tool to clear the superior orbital rim of a depressed eye;
the angle also being sufficient to dispose a tip of the tool in an orientation to aim the pulsed fluid effectively at the cataract material; and
the angle being 2° to 45°.

6. The tool of claim 5 wherein the angle is established by a shaft receiving socket in the handle.

7. The tool of claim 5 wherein the angle is achieved by a bend in the shaft near the handle.

8. A tool having a hollow needle shaft that delivers pulsed fluid against cataract material for removal from an eye, the needle shaft being oriented to aim the pressurized fluid against the cataract material to effectively break the material up for aspiration from the eye, the needle shaft comprising:
the needle shaft being oriented at an angle from the handle to allow the needle shaft to clear the superior orbital rim of a recessed eye; and
the needle shaft being angled in a handle region to dispose a tip of the needle shaft in either a right hand direction or a left hand direction to fit the needs of a surgeon manipulating the shaft.

9. The tool of claim 8 wherein an axial angle between the handle and the needle shaft is accomplished by a bend in the shaft near the handle.

10. The tool of claim 8 wherein the angle between the handle and the hollow needle shaft is established by a socket receiving the needle shaft in the handle.

11. The tool of claim 8 wherein the angle is 2° to 45°.
